(19) United States
(12) Patent Application Publication
(10) Pub. No.: US 2024/0425800 A1
(43) Pub. Date: Dec. 26, 2024

(54) METHODS AND SYSTEMS FOR SEPARATING OIL FROM ONE OR MORE STILLAGE COMPOSITIONS

(71) Applicant: POET Research, Inc., Sioux Falls, SD (US)

(72) Inventors: Cory James Sarks, Sioux Falls, SD (US); Brett A. Flittie, Harrisburg, SD (US)

(21) Appl. No.: 18/746,445

(22) Filed: Jun. 18, 2024

Related U.S. Application Data
(60) Provisional application No. 63/522,276, filed on Jun. 21, 2023.

(51) Int. Cl. C12M 1/00; B01D 21/26; B04B 1/04; B04B 5/10

(52) U.S. Cl. CPC C12M 47/10; B01D 21/262; B04B 1/04; B04B 5/10

(57) ABSTRACT

Systems and methods for separating oil from one or more stillage compositions in a bioprocessing facility.

### CROSS-REFERENCE TO RELATED APPLICATION

This nonprovisional patent application claims the benefit of U.S. Provisional Patent Application Ser. No. 63/522,276, filed on Jun. 21, 2023, wherein said provisional patent application is incorporated herein by reference in its entirety.

### BACKGROUND

The present disclosure relates to methods and systems for processing one or more stillage compositions to separate oil. There is a continuing need for improved methods and systems for processing one or more stillage compositions generated at a bioprocessing facility to separate additional oil and/or increase oil yield from the one or more stillage compositions.

### SUMMARY

The present disclosure includes embodiments of a bioprocessing facility configured to separate oil product from one or more stillage compositions in a bioprocessing facility. The bioprocessing facility includes a first separator system in fluid communication with a source of a stillage composition. The stillage composition is chosen from thin stillage or a stillage composition derived from thin stillage. The first separator system is configured to separate a first emulsion from the stillage composition. The bioprocessing facility also includes a second separator system configured to separate a first oil product from at least a portion of the first emulsion to form at least a defatted emulsion. The bioprocessing facility also includes a third separator system configured to separate at least defatted, defatted emulsion, and a second oil product or a second emulsion from at least a portion of the defatted emulsion.

The present disclosure also includes embodiments of a method of separating oil product from one or more stillage compositions in a bioprocessing facility. The method includes separating a first emulsion from a stillage composition. The stillage composition is chosen from thin stillage or a stillage composition derived from thin stillage. The method also includes separating a first oil product from at least a portion of the first emulsion to form at least a defatted emulsion; and separating at least defatted, defatted emulsion, and a second oil product or a second emulsion from at least a portion of the defatted emulsion.

The present disclosure also includes embodiments of a bioprocessing facility configured to separate oil product from one or more defatted stillage compositions in a bioprocessing facility. The bioprocessing facility includes a source of one or more enzymes and/or a source of one or more microorganisms in fluid communication with the one or more defatted stillage compositions. The bioprocessing facility is configured to combine the one or more enzymes with the one or more defatted stillage compositions and expose the one or more defatted stillage compositions to conditions to enzymatically hydrolyze one or more substrates in the one or more defatted stillage compositions and/or to combine the one or more microorganisms with the one or more defatted stillage compositions and expose the one or more defatted stillage compositions to conditions to convert residual carbohydrate in the one or more defatted stillage compositions into a fermented composition comprising one or more bioproducts. The bioprocessing facility also includes a separator system in fluid communication with at least a portion of the fermented composition. The separator system is configured to separate at least oil product or an emulsion from the at least a portion of the fermented composition.

The present disclosure also includes embodiments of a method of separating oil product from one or more defatted stillage compositions in a bioprocessing facility. The method includes combining one or more enzymes with one or more defatted stillage compositions and/or combining one or more microorganisms with the one or more defatted stillage compositions. The method also includes exposing the one or more defatted stillage compositions to conditions to enzymatically hydrolyze one or more substrates in the one or more defatted stillage compositions and/or to conditions to convert residual carbohydrate in the one or more defatted stillage compositions into a fermented composition comprising one or more bioproducts. The method also includes separating at least oil product or an emulsion from at least a portion of the fermented composition.

### BRIEF DESCRIPTION OF THE DRAWINGS

Various examples of the present disclosure will be discussed with reference to the appended drawings. These drawings depict only illustrative examples of the disclosure and are not to be considered limiting of its scope.

FIG. 1 shows a process-flow schematic of a bioprocessing facility configured to separate oil from one or more stillage compositions;

FIG. 2A shows a process-flow schematic of a non-limiting embodiment of a bioprocessing facility configured to separate oil from one or more stillage compositions according to the present disclosure;

FIG. 2B shows a process-flow schematic of a non-limiting optional embodiment of the bioprocessing facility shown in FIG. 2A;

FIG. 3 shows a process-flow schematic of another non-limiting embodiment of a bioprocessing facility configured to separate oil from one or more stillage compositions according to the present disclosure;

FIG. 4 shows a process-flow schematic of another non-limiting embodiment of a bioprocessing facility configured to separate oil from one or more stillage compositions according to the present disclosure;

FIG. 5 shows a process-flow schematic of another non-limiting embodiment of a bioprocessing facility configured to separate oil from one or more stillage compositions according to the present disclosure;

FIG. 6 shows a process-flow schematic of another non-limiting embodiment of a bioprocessing facility configured to separate oil from one or more stillage compositions according to the present disclosure; and

FIG. 7 shows side-by-side photographs of defatted syrup (left) and fermented, defatted syrup (right) after being centrifuged at 4200 RPM for 10 minutes.

### DETAILED DESCRIPTION

The present disclosure provides systems and methods for separating oil from one or more stillage compositions (also referred to as stillage “streams”). As used herein, “oil product” (or “oil”) refers to a composition or process stream that is mostly oil, like distiller's corn oil, and that has been separated from a back-end process stream (e.g., stillage) at a bioprocessing facility. While “oil product” as used herein can be an output (e.g., a saleable product) of the bioprocessing facility, an oil product does have to be an output of the bioprocessing facility. For example, an oil product could be used as an “intermediate” and introduced into another process stream within the bioprocessing facility as an input.

An oil product includes at least a triglyceride component having one or more triglycerides. In some embodiments, the triglyceride component can be present in an amount of at least 70 percent by total weight of the oil product, at least 80 by total weight of the oil product, at least 90 percent by total weight of the oil product, or even at least 95 percent by total weight of the oil product. In some embodiments, the triglyceride component can be present in an amount from 70 to 99 percent by total weight of the oil product, from 70 to 99 percent by total weight of the oil product, from 80 to 95 percent by total weight of the oil product, or even from 85 to 95 percent by total weight of the oil product. Triglycerides can be determined by test method AOCS Cd 11d-96.

In some embodiments, an oil product includes a diglyceride component having one or more diglycerides. In some embodiments, the diglyceride component can be present in an amount of 30 percent or less by total weight of the oil product, 20 percent or less by total weight of the oil product, 10 percent or less by total weight of the oil product, or even 5 percent or less by total weight of the oil product. In some embodiments, the diglyceride component can be present in an amount from 1 to 20 percent by total weight of the oil product, from 1 to 15 percent by total weight of the oil product, from 1 to 10 percent by total weight of the oil product, or even from 1 to 5 percent by total weight of the oil product. Diglycerides can be determined by test method AOCS Cd 11d-96.

In some embodiments, an oil product includes a monoglyceride component having one or more monoglycerides. In some embodiments, the monoglyceride component can be present in an amount of 20 percent or less by total weight of the oil product, 15 percent or less by total weight of the oil product, 10 percent or less by total weight of the oil product, or even 5 percent or less by total weight of the oil product. In some embodiments, the monoglyceride component can be present in an amount from 1 to 15 percent by total weight of the oil product, from 1 to 10 percent by total weight of the oil product, from 1 to 5 percent by total weight of the oil product, or even from 0.1 to 5 percent by total weight of the oil product. Monoglycerides can be determined by test method AOCS Cd 11d-96.

In some embodiments, an oil product includes a moisture content of 30 percent or less by total weight of the oil product, 20 percent or less by total weight of the oil product, 10 percent or less by total weight of the oil product, 5 percent or less by total weight of the oil product, or even 1 percent or less by total weight of the oil product. In some embodiments, the moisture content can be from 0.01 to 10 percent by total weight of the oil product, from 0.01 to 5 percent by total weight of the oil product, from 0.01 to 1 percent by total weight of the oil product, or even from 0.1 to 1 percent by total weight of the oil product. Moisture content can be determined by a Karl Fischer titration (e.g., following ASTM E1064-12 or AOCS 2e-84). As used herein, an “emulsion” refers to a mixture of two or more liquids that are usually immiscible but under certain conditions instead have a macroscopic homogeneous aspect and a microscopic heterogeneous aspect. In an emulsion, one liquid is dispersed in the other. In the present disclosure, and emulsion includes at least grain oil (e.g., corn oil) and water.

As used herein, a bioprocessing facility refers to a facility that can produce one or more bioproducts by converting biomass feedstock via one or more physical processes, one or more chemical processes, one or more bioprocesses, and combinations thereof. Non-limiting examples of bioprocessing facilities include dry mills, wet mills, biofuel production facilities, soy processing facilities, and the like. A bioproduct refers to a product derived from a biological, renewable resource. For example, a bioproduct can be a component of biomass feedstock that is liberated from the biomass feedstock (e.g., corn oil from corn grain) and/or can include a chemical (“biochemical” or “target biochemical”) that is produced by a biocatalyst (e.g., microorganism and/or enzyme) such as, for example, alcohol produced by yeast fermenting sugar. Non-limiting examples of bioproducts produced in a bioprocessing facility include one or more of fuel, feed, food, pharmaceuticals, beverages and precursor chemicals. In some embodiments, a bioproduct includes, among others, one or more monomeric sugars, one or more enzymes, one or more oils, one or more alcohols (e.g., ethanol, butanol, and the like), fungal biomass, amino acids, and one or more organic acids (e.g., lactic acid), and combinations thereof.

As used herein, a “stillage composition” refers to a back-end composition of a fermentation process after separating (e.g., via distillation) one or more bioproducts from a post-fermentation broth (e.g., beer) to form at least one target bioproduct stream (e.g., ethanol) and one or more co-product streams (e.g., whole stillage). A stillage composition can include whole stillage, at least one stillage composition derived from whole stillage, and combinations thereof. Non-limiting examples of a stillage composition derived from whole stillage include thin stillage, concentrated thin stillage (syrup), defatted syrup, defatted emulsion, clarified thin stillage, distiller's oil, distiller's grain, distiller's yeast, and the like. Defatted syrup and defatted emulsion are examples of stillage compositions that remain after fat (e.g., corn oil) has been separated from syrup and emulsion, respectively, and each can be referred to as a “defatted stillage composition.”

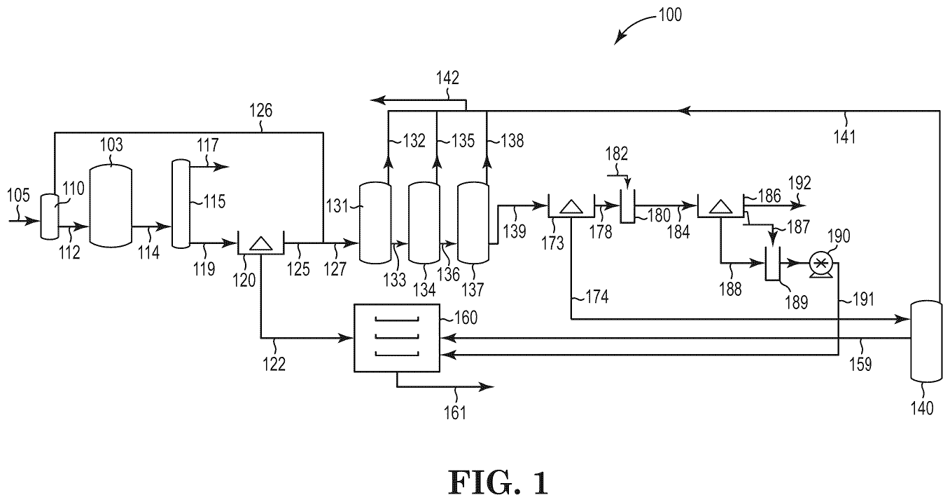

For illustration purposes, a non-limiting example of a bioprocessing facility that produces one or more stillage compositions will be described with respect to FIG. 1. FIG. 1 illustrates an example of dry-grind corn ethanol biorefinery 100 (“biorefinery 100”) as a bioprocessing facility that produces stillage compositions described above. The biorefinery 100 includes a “front end” and a “back end.” The front end includes distillation 115 and upstream from distillation 115. As shown in FIG. 1, the front end starts with adding one or more materials 105 to a slurry tank 110 to form a fermentable composition 112 (or “slurry”) that can ferment in front-end fermentation system 103. Non-limiting examples of materials 105 used to form a fermentable composition include one or more of microorganisms, enzymes, carbon sources (e.g., feedstock), aqueous compositions (e.g., fresh water, backset, etc.), nutrient sources (e.g., feedstock), etc.

In some embodiments, a feedstock can function as a carbon source and/or a nutrient source, and can be used to form a fermentable composition. A feedstock can include one or more components that are utilized by a microorganism to produce one or more bioproducts via a bioprocess. Non-limiting examples of a feedstocks can be derived from biomass (e.g., plant-based) and may include, e.g., monosaccharides such as glucose and fructose, disaccharides such as sucrose and lactose, and more complex polysaccharides such as starch, cellulose, hemicellulose, and pectin. These biomass-derived feedstocks may come from the seed, sap, stems, and leaves of plants. A wide variety of plant-based feedstocks can be used according to the present disclosure such as sugar beets, sugar cane, grains, legumes, crop residues (e.g., husks, stems, corn stover, sugarcane bagasse, wheat straw), grasses, and woody plants. In some embodiments, feedstock can be derived from corn, sorghum, wheat, rice, barley, soybean, rapeseed, oats, millet, rye, corn stover, straw, bagasse and the like. In some embodiments, as shown in FIG. 1, a feedstock can include whole ground grain (e.g., corn flour) formed via, e.g., a dry-grind process.

In some embodiments, a bioprocessing facility can include one or more feedstock systems to process feedstock from one form into another form prior to fermentation. For example, a feedstock system can include one or more size-reduction devices to reduce the size of raw feedstock such as grain and/or further reducing the size of ground grain that has previously been reduced in size. Methods for reducing the size of feedstock, e.g., corn and/or previously ground corn, into fine particles prior to fermentation include dry methods such as passing corn through one or more hammer mills, ball mills and/or roller mills or wet methods such as passing a ground grain slurry through one or more mills such as a disc mill, roller mill, colloid mill, ball mill or other type of milling device.

In some embodiments, a ground feedstock can be mixed with an appropriate amount of water (e.g., in a slurry tank 110) to form at least a portion of a fermentable composition (sometimes referred to as a mash). In some embodiments, whole ground corn can be mixed with liquid at about 20 to about 50 wt-% or about 25 to about 45 wt-% dry whole ground corn based on the total weight of the slurry. Whole ground corn can include starch, fiber, protein, oil, endogenous enzymes, amino acids, etc. As shown in FIG. 1, whole ground corn is mixed in slurry tank 110 with process water.

One or more exogenous microorganisms can be present in the fermentable composition of the fermentation system 103 to produce post-fermentation broth 114 that includes at least one or more biochemical bioproducts. A bioproduct refers to a product derived from a biological, renewable resource. For example, a bioproduct can be a component of biomass feedstock that is liberated from the biomass feedstock (e.g., corn oil from corn grain) and/or can include a chemical (“biochemical”) that is produced by a biocatalyst (e.g., microorganism and/or enzyme) such as, for example, alcohol produced by yeast fermenting sugar. Fermentation by a microorganism can produce biomass (e.g., single cell protein (SCP)), extracellular metabolites (e.g., alcohol such as ethanol), intracellular metabolites (e.g., enzymes), and combinations thereof. Non-limiting examples of such microorganisms include one or more of ethanologens, butanologens, and the like. Exemplary microorganisms include one or more of yeast, algae, or bacteria. For example, yeast may be used to convert the sugars to an alcohol such as ethanol. Suitable yeast includes any variety of commercially available yeast, such as commercial strains of Saccharomyces cerevisiae. As shown in FIG. 1, yeast is added as a microorganism to slurry tank 110 to form a fermentable composition 112.

Optionally, one or more additional, exogenous materials may be utilized in a fermentable composition. Non-limiting examples of such materials include one or more of enzymes, pH adjusters, antimicrobials (e.g., used against bacterial contaminants in yeast fermentation), and the like. As shown in FIG. 1, sulfuric acid is added to slurry tank 110 to adjust the pH, one or more antimicrobials are added to slurry tank to act against bacterial contaminants in yeast fermentation, and one or more exogenous enzymes are added discretely to slurry tank 110 to help break corn starch into monosaccharide such as glucose. Optionally or alternatively, one or more enzymes may used for hydrolysis can be produced by one or more microorganisms present in a fermentable composition.

A front-end fermentation system according to the present disclosure can include one or more vessels that are adapted to expose a fermentable composition to conditions suitable for converting monosaccharide such as glucose to one or more bioproducts. As used herein, a “vessel” refers to any vessel that permits a bioproduct to be formed from a microorganism via fermentation. In some embodiments, a vessel can refer to a bioreactor adapted or configured to expose a fermentable composition to fermentation conditions. Non-limiting examples of vessels that can expose a fermentable composition to fermentation conditions include fermenters, beer wells, and the like. Two or more vessels may be arranged in any desired configuration such as parallel or series. As shown in FIG. 1, front-end fermentation system 103 includes a single fermenter.

A front-end fermentation system is configured to expose fermentable composition to fermentation conditions so that one or more microorganisms can convert one or more components in the fermentable composition such as sugars into a post-fermentation broth that includes one or more target bioproducts. Fermentation conditions include one or more conditions such as pH, time, temperature, aeration, stirring, and the like.

The pH of a fermentable composition can be at a pH that helps a microorganism produce one or more target bioproducts in a desired quantity. In some embodiments, the pH is greater than 3.5, e.g., from 3.5 to 7, from 3.5 to 5.5, or even from 3.5 to 4.5. Techniques for adjusting and maintaining pH include, e.g., adding one or more acidic materials and/or adding one or more basic materials. As mentioned above, sulfuric acid is added to slurry tank 110 to adjust the pH.

With respect to temperature and time, the contents of a fermentable composition can be maintained at temperature for time period helps a microorganism produce one or more target bioproducts in a desired quantity. In some embodiments, the temperature of a fermentable composition can be at a temperature in a range from 20° C. to 45° F., from 25° C. to 40° C., or even from 30° C. to 40° C. In some embodiments, front-end fermentation can occur for a time period up to 72 hours, e.g., from 1 hour to 48 hours, from 2 hours to 48 hours, or even from 10 hours to 30 hours.

Front-end fermentation can be performed under anaerobic conditions and/or aerobic conditions. For example, front-end fermentation can be performed under aerobic conditions for at least a portion of the fermentation and performed under anaerobic conditions for another portion of fermentation. Alternatively, all of fermentation can be performed under anaerobic conditions or under aerobic conditions. Anaerobic or aerobic conditions are selected based on the “target” biochemical or biochemicals chosen to be produced by a microorganism even though there may de minimis amounts of “non-target” biochemicals that are also produced by the microorganism. Anaerobic conditions means that the fermentation process is conducted without any intentional introduction of oxygen-containing gases such as with equipment like blowers, compressors, etc., that could operate to create an aerobic environment suitable for aerobic fermentation. It is noted that while simply stirring a fermentable composition to keep reactor contents homogenous may or may not introduce a de minimis amount of an oxygen-containing gas such as air in some embodiments, stirring alone may not create conditions that would be considered “aerobic conditions” as used herein. However, if desired, the contents of a fermenter could be mixed using appropriate equipment such that sufficient oxygen is introduced throughout the fermentable composition to create an aerobic environment suitable for aerobic fermentation (see below).

Aerobic conditions means that fermentation is performed with intentional introduction of one or more oxygen-containing gasses (“aeration”) to create an aerobic environment suitable for aerobic fermentation so that oxygen can be consumed by one or more microorganisms and selectively favor the production of enzymes via an aerobic metabolic pathway as compared to an anaerobic pathway which favors production of biochemicals (e.g., alcohol, organic acids, and the like). A fermentation system may incorporate aeration by including one or more blowers, spargers, gas compressors, mixing devices, and the like, that are in fluid communication with one or more fermentation vessels and that can introduce an oxygen-containing gas (e.g., air) into a fermentable composition during at least a portion of fermentation. For example, an oxygen-containing gas can be sparged into a fermentable composition so that the gas bubbles up and through the fermentable composition and oxygen transfers into the fermentable composition. As another example, an oxygen-containing gas can be introduced into the headspace of a fermenter so that the gas diffuses into the fermentable composition.

In some embodiments, an aerobic fermentation can be quantified by referring to a volumetric oxygen transfer coefficient (“kLa” constant) (hours (h)-1). The kLa constant describes how efficient oxygen is transferred from gas bubbles into the fermentable composition. The kLa constant depends on factors such as process conditions and geometry of a vessel used for fermentation (e.g., a fermenter). Process conditions include the volume flow of oxygen in the form of gas into a fermentable composition, pressure of the contents of a vessel, temperature of the contents of a vessel, and/or degree of mixing of the contents of a vessel. Geometry of a vessel used for fermentation includes height of the vessel. The kLa constant consists of the two coefficients. The mass transfer coefficient “KL,” which describes the transport of oxygen and gas into the liquid phase. And “a,” which refers to the gas-liquid exchange area per unit of liquid volume. Since it can be difficult to measure the kL and a value separately, they are combined into one parameter, the kLa constant. There are chemical, biological and physical methods that measure the kLa constant in a vessel used for fermentation. One method is referred to as the “static gassing-out” method, which involves installing an oxygen sensor in a vessel used for fermentation to measure the dissolved oxygen concentration in a liquid medium. The characterization is often done with water, but any liquid medium can be used. The oxygen concentration of the liquid medium is set to zero by degassing with nitrogen. Then, oxygen containing gas is introduced or “gassed” (e.g., sparged) into the contents of the vessel again under process conditions using a defined gassing rate and stirrer speed. The oxygen sensor then measures the saturation process and the kLa can be determined. In some embodiments, a fermentation vessel operating under aerobic conditions has a kLa constant greater than 0.2, greater than 0.25 or even greater than 0.3 (e.g., from 0.3 to 5, or even from 0.35 to 3).

Optionally, in addition to aeration, a fermentable composition can be agitated or mixed to facilitate transferring oxygen into and throughout the fermentable composition so as to achieve an aerobic environment. For example, a continuous stirred tank reactor (CSTR) can be used to agitate or mix the fermentable composition. The speed of the stirring mechanism (rpms) can be adjusted based on a variety of factors such as tank size, viscosity, and the like. As mentioned above, in addition to mixing the contents of a composition, mixing can be selected, if desired, to intentionally incorporate oxygen to a fermentable composition to facilitate aerobic fermentation.

A front-end fermentation system can be operated according to batch fermentation, fed-batch fermentation, or continuous fermentation (continuous feed and discharge from a vessel such as a fermenter).

Also, a front-end fermentation system according to the present disclosure can conduct fermentation sequentially or simultaneously with respect to a polysaccharide hydrolysis/saccharification process (e.g., jet-cooking and/or enzymatic hydrolysis). Saccharification and fermentation can occur simultaneously according to what is known as “simultaneous saccharification and fermentation” (“SSF”). Sequential hydrolysis and fermentation can also be referred to as separate hydrolysis and fermentation (SHF).

An example of an SSF is described below in the context of a starch-containing grain such as corn. A slurry (“grain mash composition”) can be combined with a microorganism to form a fermentable composition so that at least a portion of starch in the fermentable composition is hydrolyzed by one or more enzymes to produce monosaccharides. As the monosaccharides are produced, they can be metabolized by a microorganism into a target biochemical product. For example, sugar (glucose, xylose, mannose, arabinose, etc.) that is generated from saccharification can be fermented into one or more biochemicals (e.g., butanol, ethanol, and the like).

Alternatively, an SHF process may include a dedicated saccharification process that is separate from a fermentation process (either in the same or separate vessel). For example, after forming an aqueous slurry that includes the biomass feedstock (e.g. corn material from a milling system) the aqueous slurry can be subjected to saccharification in one or more slurry tanks to break down (hydrolyze) at least a portion of the polysaccharides, e.g. starch, cellulose, hemicellulose, etc., into oligosaccharides and/or monosaccharides (e.g. glucose, xylose, mannose, arabinose, etc.) that can be used by microorganisms (e.g., yeast) in a subsequent fermentation process.

Saccharification can be performed by a variety of mechanisms. For example, heat and/or one or more enzymes can be used to form one or more monosaccharides by saccharifying one or more oligosaccharides and/or one or more polysaccharides that are present in a polysaccharide such as starch. In some embodiments, a relatively low temperature saccharification process (whether used in SSF or SHF) involves enzymatically hydrolyzing at least a portion of starch in an aqueous slurry at a temperature below starch gelatinization temperatures, so that saccharification occurs directly from the raw native insoluble starch to soluble glucose while bypassing conventional starch gelatinization conditions, which are typically in a range of 57° C. to 93° C. depending on the starch source and polysaccharide type. In some embodiments, saccharification includes using one or more enzymes (e.g., alpha-amylases and/or gluco-amylases) to enzymatically hydrolyze at least a portion of the starch in the aqueous slurry at a temperature of 40° C. or less to produce a slurry that includes glucose. In some embodiments, enzymatic hydrolysis occurs at a temperature in the range of from 25° C. to 35° C. to produce a slurry that includes glucose. Non-limiting examples of converting starch to glucose are described in U.S. Pat. No. 7,842,484 (Lewis), U.S. Pat. No. 7,919,289 (Lewis), U.S. Pat. No. 7,919,291 (Lewis et al.), U.S. Pat. No. 8,409,639 (Lewis et al.), U.S. Pat. No. 8,409,640 (Lewis et al.), U.S. Pat. No. 8,497,082 (Lewis), U.S. Pat. No. 8,597,919 (Lewis), U.S. Pat. No. 8,748,141 (Lewis et al.), 2014-0283226 (Lewis et al.), and 2018-0235167 (Lewis et al.), wherein the entirety of each patent document is incorporated herein by reference.

After fermentation, one or more bioproducts can be separated from post-fermentation broth 114 to form at least one target bioproduct stream (e.g., ethanol) and one or more co-product streams (e.g., whole stillage). A separation system according to the present disclosure can be configured to separate at least a portion of at least one of the one or more bioproducts produced in the fermentation system 103. Referring to FIG. 1, an exemplary separation system is illustrated as a distillation system 115 in fluid communication with the fermentation system 103 to receive a post-fermentation broth 114, which is separated into ethanol 117 as a target biochemical and whole stillage 119. Whole stillage includes protein (e.g., plant protein (e.g., corn protein) and/or yeast protein from spent yeast cells), oil, fiber, residual carbohydrate (e.g., cellulose, starch, and the like), vitamins, minerals, and combinations thereof.

Whole stillage 119 can be separated into thin stillage and wet cake using one or more solid-liquid separators 120. Non-limiting examples of solid-liquid separators include one or more centrifuges (e.g., two-phase vertical disk-stack centrifuge, three-phase vertical disk-stack centrifuge, filtration centrifuge), one or more decanters (e.g., filtration decanters), one or more filters (e.g., fiber filter, rotary vacuum drum filter, filter device having one or more membrane filters), one or more screen devices (e.g., a “DSM” screen; one or more pressure screens; one or more paddle screens; one or more rotary drum screens; one or more centrifugal screeners; one or more linear motion screens; one or more vacu-deck screen; etc.), one or more brush strainers, one or more vibratory separators, one or more hydrocyclones, and combinations thereof. As shown in FIG. 1, whole stillage is fed to decanter 120, or multiple decanters in parallel, to separate whole stillage 119 into wet cake 122 and thin stillage 125. The wet cake 122 is dried in dryer system 160 to form dried distillers' grain (DDGS) 161.

A portion 126 of the thin stillage 125 is transferred to the slurry tank 110 as backset, while the rest 127 of the thin stillage 125 is transferred to an evaporation train that may include 4 to 8 evaporators in series (depending on plant size) to remove water and form syrup. As shown in FIG. 1, the evaporation train includes four evaporators 131, 134, 137, and 140. The rest 127 of the thin stillage 125 is first fed to evaporator 131 to separate moisture as water vapor 132 and form a concentrated thin stillage 133. The concentrated thin stillage 133 is fed to evaporator 134 to separate moisture as water vapor 135 and form a concentrated thin stillage 136. The concentrated thin stillage 136 is fed to evaporator 137 to separate moisture as water vapor 138 and form a semi-concentrated syrup 139. Prior to reaching the end of the evaporator train the semi-concentrated syrup (“skim feed”) 139 is sent to a corn oil separation system, which removes corn oil product 192.

First, the skim feed 139 is separated in a “skim” centrifuge 173 into an emulsion 178 and defatted syrup 174. The defatted syrup 174 is sent to the final evaporator 140 in the evaporator train to separate moisture as water vapor 141 to form syrup 159, which is sent to dryer 160 along with wet cake 122 to form DDGS 161. The emulsion 178 is combined with caustic 182 in emulsion tank 180 to help “break” the emulsion into an oil phase and aqueous phase that are more easily separated from each other. The treated emulsion 184 is pumped to oil centrifuge 186, wherein the treated emulsion 184 is separated into a corn oil product 192 and defatted emulsion (DFE) 188. The defatted emulsion 188 can accumulate in defatted emulsion (DFE) tank 189 and defatted emulsion 191 can be pumped via pump 190 to any desired location. As shown in FIG. 1, the defatted emulsion 191 is sent to dryer 160 along with wet cake 122 and syrup 159 to form DDGS 161. Also, all of the water vapor streams 132, 135, 138 and 141 are combined into a single distillate stream 142.

The skim centrifuge 173 and oil centrifuge 186 can be centrifuges such as disk-stack centrifuges. In some embodiments, the skim centrifuge 173 and/or the oil centrifuge 186 can be configured to continuously or intermittently discharge accumulated solid particles (referred to as “discharges”). As shown in FIG. 1, discharges 187 from oil centrifuge 186 are combined with defatted emulsion 188 in DFE tank 189.

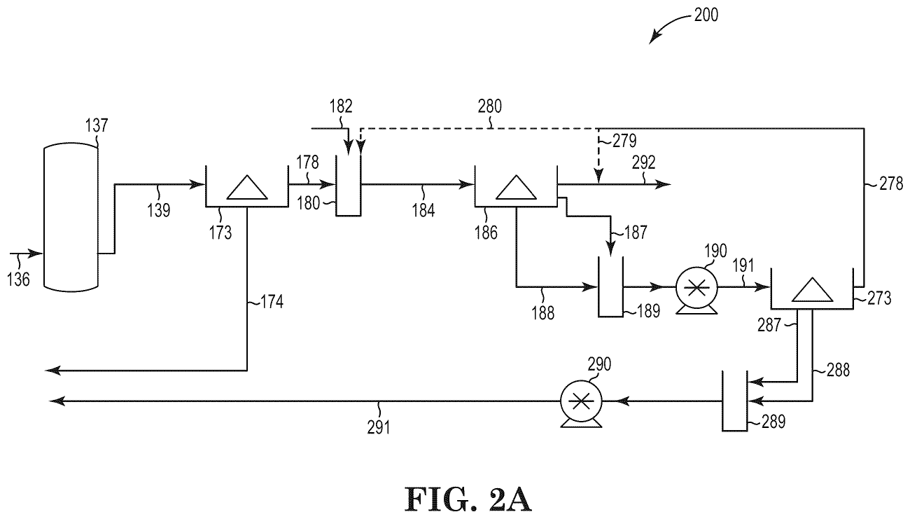

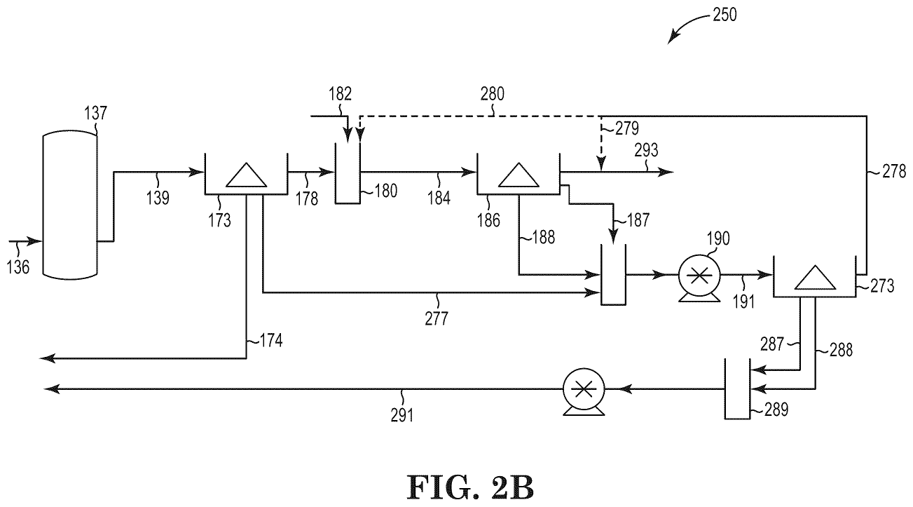

FIGS. 2A-6 are process flow diagrams showing non-limiting embodiments of the present disclosure. FIGS. 2A-6 include portions of FIG. 1 along with repeated reference characters, the discussion of which may or may not be repeated. FIGS. 2A-6 may also omit portions of FIG. 1 such as the front end of FIG. 1 for the sake of brevity.

According to an aspect of the present disclosure, methods and systems are provided that increase oil yield by separating oil from one or more defatted stillage compositions derived from thin stillage. Non-limiting examples of defatted stillage compositions derived from thin stillage include defatted syrup, defatted emulsion, and the like.

Non-limiting examples of process flow diagrams showing how residual oil can be separated from defatted emulsion is illustrated in FIGS. 2A and 2B.

FIG. 2A illustrates how a biorefinery 200 is configured to separate additional oil from the defatted emulsion 191 instead of forming DDGS 161 with defatted emulsion 191. As shown in FIG. 2A, defatted emulsion 191 is sent to a separator system configured to separate at least a portion of the defatted emulsion 191 into at least stream 278 and a defatted, defatted emulsion 288 (“double DFE”). In some embodiments, stream 278 can surprisingly be a corn oil product 279 like corn oil product 292. As shown, if desired, corn oil product 279 can be combined with corn oil product 292. In some embodiments, stream 278 can be an emulsion 280. Additional oil can be separated from emulsion 280 by returning emulsion 280 to emulsion tank 180 and allowing it to be processed again. While not being bound by theory, it is believed stream 278 may be discharged from DFE centrifuge 273 as corn oil product 279 depending on how well oil centrifuge 186 is separating oil from treated emulsion 184. For example, oil centrifuge 186 may be operating in a manner that causes a relatively higher content of oil to be discharged with defatted emulsion 188 and the DFE centrifuge 273 is able to produce stream 278 that is corn oil product 279. In other cases, oil centrifuge 186 may be operating in a manner that causes a relatively lower content of oil to be discharged with defatted emulsion 188 and the DFE centrifuge 273 is operated to take a heavy cut such that stream 278 is emulsion 280 and benefits from being returned to the emulsion tank 180 for reprocessing. Also, while not being bound by theory, it is believed stream 278 may be discharged from DFE centrifuge 273 as corn oil product 279 depending on the residence time and/or g-force experienced by defatted emulsion 192 in DFE centrifuge 273. For example, because the volumetric flow rate of corn oil product 279 is relatively low (e.g., as compared to corn oil product 292), it is believed that if defatted emulsion 188 is exposed to long enough retention time and/or high enough g-force in DFE centrifuge 273, then what would be an emulsion 280 is broken such that corn oil product 279 is instead discharged from DFE centrifuge 273.

A mechanical device can be positioned in stream 278 to permit stream 278 to be directed to a desired location depending on whether stream 278 is corn oil product 279 or emulsion 280. For example, as shown in FIG. 2A, a mechanical device could direct corn oil product 279 to be combined with corn oil product 292. As another example, as shown in FIG. 2A, a mechanical device could direct emulsion 280 to emulsion tank 180. Non-limiting examples of a mechanical device for directing stream 278 include a valve, a diverter, and the like.

Referring back to FIG. 2A, the defatted emulsion 191 is separated in a DFE centrifuge 273 (e.g., disk stack centrifuge) into stream 278 and defatted, defatted emulsion 288. If stream emulsion 278 is an emulsion 280, emulsion 280 can be recycled to emulsion tank 180 along with emulsion 178 for separation of corn oil product 292 in a similar manner as described above with respect to corn oil product 192 in FIG. 1. Like skim centrifuge 173 and/or the oil centrifuge 186, the DFE centrifuge 273 can be configured to continuously or intermittently discharge accumulated solid particles as discharges 287. The discharges 287 and defatted, defatted emulsion 288 can accumulate in defatted emulsion (DFE) tank 289. The contents 291 of DFE tank 289 can be pumped via pump 290 to any desired location. For example, the contents 291 of DFE tank 289 can be sent to dryer 160 along with wet cake 122 and syrup 159 to form DDGS 161. In some embodiments, at least a portion of the contents 291 of DFE tank 289 can be sent directly to dryer 160. In some embodiments at least a portion of the contents 291 of DFE tank 289 can be combined (e.g., via a tank or pipe) with wet cake 122 and/or syrup 159 prior to entering dryer 160.

FIG. 2B shows a biorefinery 250 that is identical to biorefinery 200, except that discharges 277 from skim centrifuge 173 are combined with defatted emulsion 188 and discharges 187 from oil centrifuge 186 in DFE tank 189. While not being bound by theory, it is believed that residual oil is present in skim discharges 277 that can be separated in oil centrifuge 186 so as to increase the yield of corn oil product 293 (e.g. via emulsion 280 or if corn oil product 279 is added to corn oil product 292) as compared to corn oil product 292 and 192.

Optionally, one or more of the feed streams entering skim centrifuge 173, oil centrifuge 186, and/or DFE centrifuge 273 can have their flow rate adjusted, be diluted, be heated (e.g., to about 160-180F), and/or be treated by adding one or more demulsifiers.

According to another aspect of the present disclosure, methods and systems are provided that produce one or more bioproducts by back-end (“secondary”) fermentation of residual carbohydrate in one or more stillage compositions using one or more microorganisms while at the same time liberating trapped oil that can then be separated into an oil product, thereby increasing oil yield.

Secondary fermentation involves converting residual carbohydrate in a given stillage composition by adding one or more microorganisms and/or one or more enzymes to the stillage composition under conditions so that the microorganism can convert monosaccharide derived from the residual carbohydrate into one or more bioproducts. Advantageously, such methods and systems can recover oil that may have been ended up in one or more other process compositions, or end products such as dried distiller's grains and solubles (DDGS). As yet another advantage, the secondary fermenting process can produce one or more bioproducts that can also be recovered. Non-limiting examples of such bioproducts include one or more amino acids, one or more organic acids (e.g., butyrate), one or more alcohols, one or more proteins, one or more oils, and the like.

According to the present disclosure, the secondary fermentation is not a separation process but a conversion process in which residual carbohydrate in a stillage composition is converted to bioproduct by a microorganism. For example, with respect to protein as a bioproduct the secondary fermentation is not a process that separates protein from residual carbohydrate but a conversion process in which residual carbohydrate in a stillage composition is converted to protein by a microorganism. The theoretical maximum protein available for recovery in the fermented stillage composition is the protein in the stillage composition prior to secondary fermentation (e.g., plant (e.g., corn) protein, microorganism cell mass protein, enzyme protein, and/or non-functional peptides) plus the proteinaceous biomass produced by the secondary microorganism, including the secondary microorganism itself.

Further, fermenting residual carbohydrate in a stillage composition can reduce or remove residual carbohydrate and help avoid undue browning that may occur in downstream heating processes (e.g., evaporators and/or dryers), which can be undesirable in some animal feed products such as distiller's dried yeast (DDY) and/or distiller's dried grains with solubles (DDGS).

As mentioned, one or more exogenous enzymes can be discretely added to a stillage composition to enzymatically hydrolyze one or more substrates in a stillage composition facilitate oil liberation and/or fermentation. Also, one or more enzymes may be expressed by one or more microorganisms selected for secondary fermentation (discussed below). An enzyme can be selected for a corresponding substrate. For example, one or more amylases could be selected to enzymatically hydrolyze residual carbohydrate such as starch. Non-limiting examples of such enzymes include one or more cellulases, one or more hemicellulases, one or more pectinases, one or more glucanases, one or more xylanases, one or more amylases, one or more proteases, one or more lipases, one or more esterases, one or more phytases, one or more monooxygenases (e.g., one or more lytic polysaccharide monooxygenases (LPMOs)), one or more peroxidases, one or more laccases, and combinations thereof. Non-limiting examples of enzymatically hydrolyzing residual carbohydrate are disclosed in U.S. Pat. Nos. 7,842,484 and 7,919,291, wherein each patent is hereby incorporated by reference in its entirety.

One or more microorganisms can be used to in secondary fermentation to convert residual carbohydrate into one or more bioproducts as described herein. In some embodiments, the microorganism can tolerate the temperatures (are thermophilic) typically present in a given stillage composition such that the stillage composition does not have to be cooled from its otherwise normal operating temperature for the fermentation to occur. In some embodiments, a microorganism selected for secondary fermentation can be can be a strain that is aerobically fermented (“propagated”) to grow and reproduce to increase the cell mass of the microorganism, which can be used to make a high protein product such as dried distiller's yeast (DDY) and/or added to dried distiller's grain with solubles (DDGS). In some embodiments, a microorganism selected for secondary fermentation can be a strain that produces one or more amino acids. Producing amino acids in the secondary fermentation can be used to form new and more valuable feed products such as dried distiller's yeast (DDY) and/or dried distiller's grain with solubles (DDGS).

Non-limiting examples of microorganisms suitable for fermenting residual carbohydrate as described herein includes at least strain of Candida utilis, at least one strain of Saccharomyces cerevisiae, at least strain of Bacillus subtilis, at least strain of Escherichia coli, at least strain of Aspergillus niger, at least strain of Kluyveromyces marxianus, at least strain of Yarrowia lipolytica, at least strain of Candida tropicalis, at least strain of Candida albicans, and combinations thereof. It is noted that Candida utilis can be desirable as a protein source in an animal feed and/or animal feed supplement.

The one or more microorganisms used in front-end fermentation can be the same or different from one or more microorganisms used in back-end (secondary) fermentation. In some embodiments, two or more different microorganisms can be selected for secondary fermentation (also referred to as “co-culturing”). This may be desirable for one or more reasons. For example, in some embodiments, each microorganism could produce one or more desirable bioproducts. Non-limiting examples include: 1) each microorganism could produce one or more different amino acids; and 2) one or more microorganisms could be a protein specific strain, one or more microorganisms could be an amino acid specific strain, and one or more microorganisms could be an organic acid (e.g., butyric acid) specific strain, etc. As another example, in some embodiments, one or more microorganisms could consume one or more byproducts of one or more different microorganisms. One microorganism could produce an amino acid and one or more byproducts (e.g., organic acid, alcohol, and the like) and another microorganism could consume at least one of the byproducts.

Secondary fermentation can be aerobic or anaerobic as discussed above with respect to front-end fermentation.

Secondary fermentation can be performed in a sequential process that involves first enzymatically hydrolyzing residual carbohydrate (saccharification) followed by fermentation, or simultaneous saccharification and fermentation (SSF).

Post-fermentation broth produced by secondary fermentation can be processed to separate liberated oil, thereby increasing oil yield as compared to if secondary fermentation had not been performed. In some embodiments, one or more bioproducts produced by secondary fermentation can be separated from post-fermentation broth.

Post-fermentation broth produced by secondary fermentation can be separated into two or more process streams using a separator system that includes one or more solid-liquid separators. Non-limiting examples of solid-liquid separators include one or more centrifuges (e.g., two-phase vertical disk-stack centrifuge, three-phase vertical disk-stack centrifuge, filtration centrifuge), one or more decanters (e.g., filtration decanters), one or more filters (e.g., fiber filter, rotary vacuum drum filter, filter device having one or more membrane filters), one or more screen devices (e.g., a “DSM” screen; one or more pressure screens; one or more paddle screens; one or more rotary drum screens; one or more centrifugal screeners; one or more linear motion screens; one or more vacu-deck screen; etc.), one or more brush strainers, one or more vibratory separators, one or more hydrocyclones, and combinations thereof. In some embodiments, a separator system is configured to separate the post-fermentation broth produced by secondary fermentation (fermented composition) into at least an emulsion and a defatted fermented composition, and at least a portion of the defatted fermented composition is in fluid communication with an evaporator system and/or a dryer system.

In some embodiments, post-fermentation broth produced by secondary fermentation can be in fluid communication with an evaporator system configured to condense the at least a portion of the post-fermentation broth prior to a solid-liquid separator.

In some embodiments, at least a portion of the output of a secondary fermentation according to the present disclosure can have an increased ash content. If desired, the ash content may be reduced by, e.g., adding a “filler” material such as DDGS to reduce the concentration of the ash and/or at least a portion of post-fermentation broth from secondary fermentation can be treated or processed to remove ash.

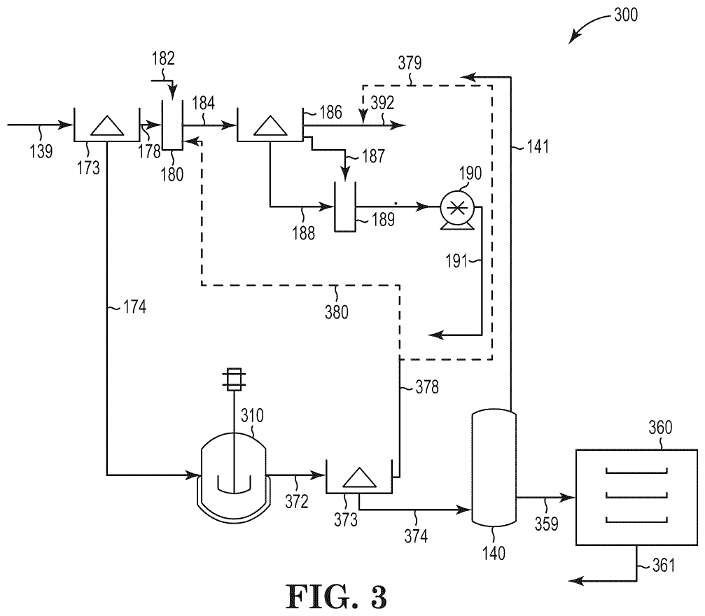

Non-limiting examples of process flow diagrams showing how residual oil can be liberated from a stillage composition via at least secondary fermentation to increase oil yield are illustrated in each of FIGS. 3-7. In some embodiments, if desired, only enzymatic hydrolysis could be used instead of secondary fermentation to liberate residual oil from a stillage composition to increase oil yield. In such embodiments, the secondary fermentation illustrated in each of FIGS. 3-6, could be substituted with only enzymatic hydrolysis.

FIG. 3 illustrates how a biorefinery 300 is configured to liberate additional oil from the defatted syrup 174 by feeding defatted syrup 174 to fermentation system 310 to form fermented syrup 372. At the same time, secondary fermentation in fermentation system 310 can produce one or more desirable bioproducts, if desired. It has been observed that while defatted syrup 174 may contain additional fat (oil), the oil that is present is not amenable to being directly separated by centrifugation. While not being bound by theory, it is believed that at least a portion of the oil present in defatted syrup 174 is “trapped” or bound up. According to the present disclosure, defatted syrup 174 can be exposed to fermentation conditions to liberate the trapped oil so that the liberated oil can be separated into an oil product, thereby increasing the oil yield as compared to if defatted syrup 174 had not been exposed to fermentation conditions. Bioprocessing facility 300 is configured to combine at least one or more microorganisms with the defatted syrup 174 and expose the defatted syrup in fermenter 310 to conditions to convert residual carbohydrate in the defatted syrup 174 into a fermented syrup 372. As shown in FIG. 3, fermentation is performed under aerobic conditions. Optionally, bioprocessing facility 300 can be configured to discretely combine one or more exogenous enzymes with the defatted syrup 174 and expose the defatted syrup 174 to conditions to enzymatically hydrolyze one or more substrates in the defatted syrup to help liberate oil. A separator system 373 is in fluid communication with fermented syrup 372 and is configured to separate a stream 378 from the fermented syrup 372. As similarly discussed above with respect to stream 278 in FIG. 2, stream 378 can be a corn oil product 379 or an emulsion 380. If stream 378 is an emulsion 380, additional oil can be separated from emulsion 380. As shown in FIG. 3, the fermented syrup 372 is separated in a centrifuge 373 (e.g., disk stack centrifuge) into stream 378 and defatted fermented syrup 374. If stream 278 is an emulsion 380, the emulsion 380 can be recycled to emulsion tank 180 along with emulsion 178 for separation of corn oil product 392 in a similar manner as described above with respect to corn oil product 192 in FIG. 1. Otherwise, corn oil product 379 can be combined with corn oil product 392, if desired. A mechanical device can be positioned in stream 378 to permit stream 378 to be directed to a desired location depending on whether stream 378 is corn oil product 379 or emulsion 380. For example, as shown in FIG. 3, a mechanical device could direct corn oil product 379 to be combined with corn oil product 392. As another example, as shown in FIG. 3, a mechanical device could direct emulsion 380 to emulsion tank 180. Non-limiting examples of a mechanical device for directing stream 378 include a valve, a diverter, and the like.

Like skim centrifuge 173 and/or the oil centrifuge 186, the centrifuge 373 can be configured to continuously or intermittently discharge accumulated solid particles as discharges. The defatted, fermented syrup 374 can be condensed in evaporator 140 of the evaporator train to form a concentrated, defatted, fermented syrup 359. The concentrated, defatted, fermented syrup 359 can be pumped to any desired location depending on, e.g., the one or more microorganisms selected for secondary fermentation. As shown in FIG. 3, concentrated, defatted, fermented syrup 359 is sent to dryer 360 to form fermented syrup product 361 including one or more bioproducts (e.g., protein and/or amino acids) produced by the aerobic, secondary fermentation performed in fermenter 310.

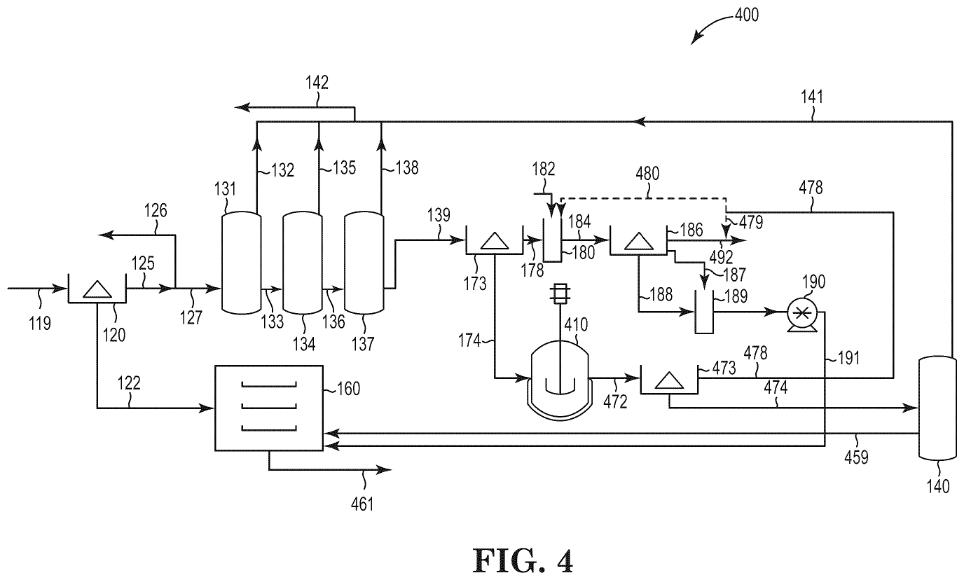

FIG. 4 is similar to FIG. 3, except that defatted, fermented syrup is used to make enhanced DDGS 461 instead of a fermented syrup product 361. The DDGS 461 is considered enhanced compared to DDGS 161 because of one or more bioproducts (e.g., protein and/or amino acids using an amino acid producing strain microorganism) produced by the aerobic, secondary fermentation performed in fermenter 410. In more detail, FIG. 4 illustrates how a biorefinery 400 is configured to liberate additional oil from the defatted syrup 174 by feeding defatted syrup 174 to fermentation system 410 to form fermented syrup 472 while at the same time producing one or more bioproducts that can help form enhanced DDGS 461. Bioprocessing facility 400 is configured to combine at least one or more microorganisms with the defatted syrup 174 and expose the defatted syrup in fermenter 410 to conditions to convert residual carbohydrate in the defatted syrup 174 into a fermented syrup 472. As shown in FIG. 4, fermentation is performed under aerobic conditions. Optionally, bioprocessing facility 400 can be configured to discretely combine one or more exogenous enzymes with the defatted syrup 174 and expose the defatted syrup 174 to conditions to enzymatically hydrolyze one or more substrates in the defatted syrup to help liberate oil. A separator system 473 is in fluid communication with fermented syrup 472 and is configured to separate stream 478 from the fermented syrup 472. As similarly discussed above with respect to stream 278 in FIG. 2, stream 478 can be a corn oil product 479 or an emulsion 480. If stream 478 is an emulsion 480, additional oil can be separated from emulsion 480. As shown in FIG. 4, the fermented syrup 472 is separated in a centrifuge 473 (e.g., disk stack centrifuge) into stream 478 and defatted fermented syrup 474. If stream 478 is an emulsion 480, the emulsion 480 can be recycled to emulsion tank 180 along with emulsion 178 for separation of corn oil product 492 in a similar manner as described above with respect to corn oil product 192 in FIG. 1. Otherwise, corn oil product 479 can be combined with corn oil product 492, if desired. A mechanical device can be positioned in stream 478 to permit stream 478 to be directed to a desired location depending on whether stream 478 is corn oil product 479 or emulsion 480. For example, as shown in FIG. 4, a mechanical device could direct corn oil product 479 to be combined with corn oil product 492. As another example, as shown in FIG. 4, a mechanical device could direct emulsion 480 to emulsion tank 180. Non-limiting examples of a mechanical device for directing stream 478 include a valve, a diverter, and the like.

Like skim centrifuge 173 and/or the oil centrifuge 186, the centrifuge 473 can be configured to continuously or intermittently discharge accumulated solid particles as discharges. The defatted, fermented syrup 474 can be condensed in evaporator 140 of the evaporator train to form a concentrated, defatted, fermented syrup 459. The concentrated, defatted, fermented syrup 459 can be pumped to any desired location depending on, e.g., the one or more microorganisms selected for secondary fermentation. As shown in FIG. 4, the defatted emulsion 191 is sent to dryer 160 along with wet cake 122 and concentrated, defatted, fermented syrup 459 to form enhanced DDGS 461.

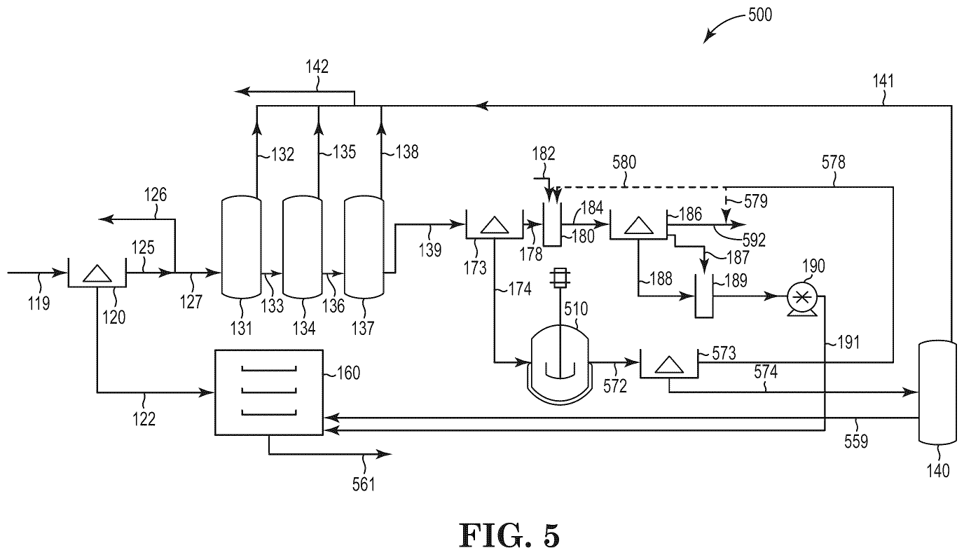

FIG. 5 is similar to FIG. 4, except that secondary fermentation in fermenter 510 is performed under anaerobic conditions instead of aerobic conditions, which may produce DDGS 561 that is more similar to DDGS 161 than DDGS 461 depending on the one or more microorganisms selected for secondary fermentation and the corresponding bioproducts produced. In more detail, FIG. 5 illustrates how a biorefinery 500 is configured to liberate additional oil from the defatted syrup 174 by feeding defatted syrup 174 to fermentation system 510 to form fermented syrup 572 while at the same time producing one or more bioproducts. Bioprocessing facility 500 is configured to combine at least one or more microorganisms with the defatted syrup 174 and expose the defatted syrup in fermenter 510 to conditions to convert residual carbohydrate in the defatted syrup 174 into a fermented syrup 572. As shown in FIG. 5, fermentation is performed under anaerobic conditions. Optionally, bioprocessing facility 500 can be configured to discretely combine one or more exogenous enzymes with the defatted syrup 174 and expose the defatted syrup 174 to conditions to enzymatically hydrolyze one or more substrates in the defatted syrup to help liberate oil. A separator system 573 is in fluid communication with fermented syrup 572 and is configured to separate stream 578 from the fermented syrup 572. As similarly discussed above with respect to stream 278 in FIG. 2, stream 578 can be a corn oil product 579 or an emulsion 580. If stream 578 is an emulsion 580, Additional oil can be separated from emulsion 580. As shown in FIG. 5, the fermented syrup 572 is separated in a centrifuge 573 (e.g., disk stack centrifuge) into stream 578 and defatted fermented syrup 574. If stream 578 is an emulsion 580, the emulsion 580 can be recycled to emulsion tank 180 along with emulsion 178 for separation of corn oil product 592 in a similar manner as described above with respect to corn oil product 192 in FIG. 1. Otherwise, corn oil product 579 can be combined with corn oil product 592, if desired. A mechanical device can be positioned in stream 578 to permit stream 578 to be directed to a desired location depending on whether stream 578 is corn oil product 579 or emulsion 580. For example, as shown in FIG. 5, a mechanical device could direct corn oil product 579 to be combined with corn oil product 592. As another example, as shown in FIG. 5, a mechanical device could direct emulsion 580 to emulsion tank 180. Non-limiting examples of a mechanical device for directing stream 578 include a valve, a diverter, and the like.

Like skim centrifuge 173 and/or the oil centrifuge 186, the centrifuge 573 can be configured to continuously or intermittently discharge accumulated solid particles as discharges. The defatted, fermented syrup 574 can be condensed in evaporator 140 of the evaporator train to form a concentrated, defatted, fermented syrup 559. The concentrated, defatted, fermented syrup 559 can be pumped to any desired location depending on, e.g., the one or more microorganisms selected for secondary fermentation. As shown in FIG. 5, the defatted emulsion 191 is sent to dryer 160 along with wet cake 122 and concentrated, defatted, fermented syrup 559 to form DDGS 561.

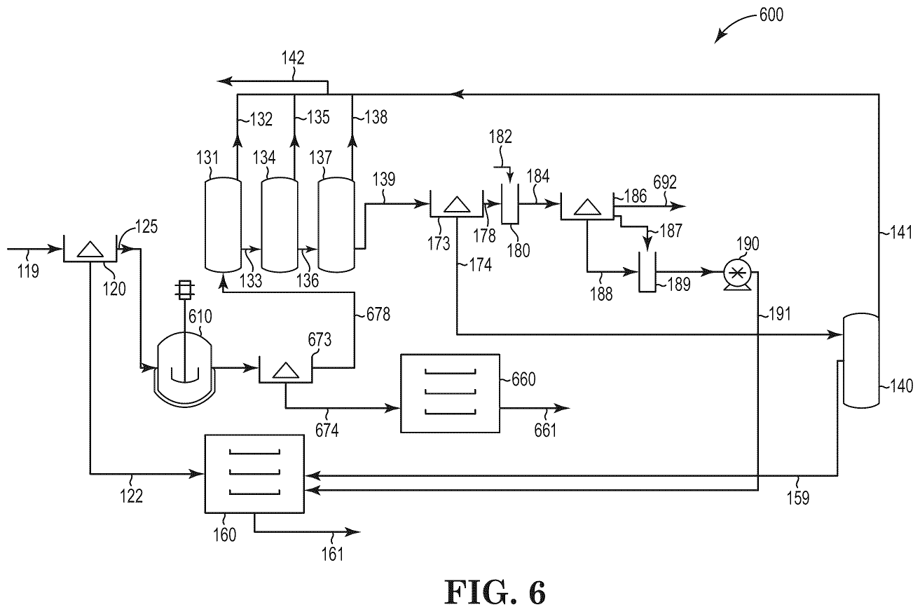

FIG. 6 is similar to FIG. 4, except that secondary fermentation is performed on thin stillage 125 instead of defatted syrup 174 to produce grain distillers dried yeast (GDDY) 661, which includes single-cell protein obtained as a co-product during secondary fermentation. GDDY 661 is considered enhanced compared to a GDDY product produced using only front-end fermentation to produce grain ethanol because GDDY 661 can further include of one or more bioproducts (e.g., protein and/or amino acids) produced by the aerobic, secondary fermentation performed in fermenter 410. In more detail, FIG. 6 illustrates how a biorefinery 600 is configured to liberate additional oil from at least a portion of thin stillage 125 by feeding at least a portion of thin stillage 125 to fermentation system 610 to form fermented thin stillage 672 while at the same time producing one or more bioproducts that can help form enhanced GDDY 661. Bioprocessing facility 600 is configured to combine at least one or more microorganisms with at least a portion of thin stillage 125 and expose the thin stillage in fermenter 610 to conditions to convert residual carbohydrate in the thin stillage into a fermented thin stillage 672. As shown in FIG. 6, fermentation is performed under aerobic conditions. Optionally, bioprocessing facility 600 can be configured to discretely combine one or more exogenous enzymes with at least a portion of the thin stillage 125 and expose the thin stillage to conditions to enzymatically hydrolyze one or more substrates in the thin stillage to help liberate oil. A separator system 673 in fluid communication with fermented thin stillage 672 and configured to separate light phase 678 from the fermented thin stillage 672. Additional oil can then be separated from light phase 678 as compared to thin stillage 127 in FIG. 1. As shown in FIG. 6, the fermented thin stillage 672 is separated in a centrifuge 673 (e.g., disk stack centrifuge) into light phase 678 and a suspended solids portion 674. The light phase 678 is sent to evaporator 131 of the evaporator train like thin stillage 127 in FIG. 1, and ultimately for separation of corn oil product 692 in a similar manner as described above with respect to corn oil product 192 in FIG. 1. Like skim centrifuge 173 and/or the oil centrifuge 186, the centrifuge 673 can be configured to continuously or intermittently discharge accumulated solid particles as discharges. The suspended solids portion 674 can be dried in dryer system 660 to form enhanced GDDY 661.

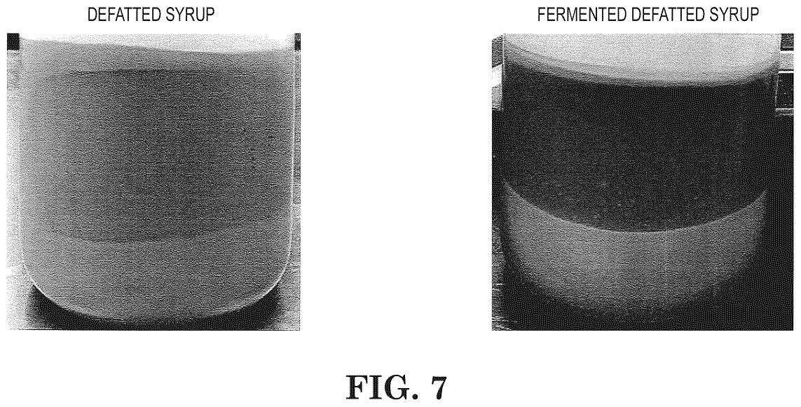

FIG. 7 shows side-by-side photographs of defatted syrup (left) and fermented, defatted syrup (right) after being centrifuged at 4200 RPM for 10 minutes.

Non-limiting embodiment of the present disclosure are as follows:

1. A bioprocessing facility configured to separate oil product from one or more stillage compositions in a bioprocessing facility, wherein the bioprocessing facility comprises:
a first separator system in fluid communication with a source of a stillage composition, wherein the stillage composition is chosen from thin stillage or a stillage composition derived from thin stillage, wherein the first separator system is configured to separate a first emulsion from the stillage composition;
a second separator system configured to separate a first oil product from at least a portion of the first emulsion to form at least a defatted emulsion; and
a third separator system configured to separate at least defatted, defatted emulsion, and a second oil product or a second emulsion from at least a portion of the defatted emulsion.
   2. The bioprocessing facility of embodiment 1, wherein the first separator system is in fluid communication with an evaporator system configured to condense thin stillage into syrup, and wherein the first separator system is configured to separate at least a portion of the syrup from the evaporator system into at least the first emulsion and defatted syrup.
   3. The bioprocessing facility of embodiment 2, wherein the first separator system comprises at least one centrifuge configured to separate at least a portion of the syrup from the evaporator system into at least the first emulsion and the defatted syrup.
   4. The bioprocessing facility of any of embodiments 2-3, wherein the second separator system comprises at least one centrifuge configured to separate at least a portion of the first emulsion from first separator system into at least the first oil product and the defatted emulsion.
   5. The bioprocessing facility of any of embodiment 2-4, wherein the third separator system configured to separate at least a portion of the defatted emulsion into at least the second emulsion and the defatted, defatted emulsion, wherein at least a portion of the second emulsion is in fluid communication with a fourth separator system configured to separate oil product from at least a portion of the second emulsion, wherein the fourth separator system is the same or different from the second separator system.
   6. The bioprocessing facility of any of embodiments 2-5, wherein the third separator system comprises at least one centrifuge.
   7. The bioprocessing facility of any of embodiments 2-6, wherein the defatted, defatted emulsion is in fluid communication with a dryer system to form a dried product.
   8. The bioprocessing facility of any of embodiments 2-7, wherein the first separator system, the second separator system, and/or the third separator system comprise at least one disk stack centrifuge.
   9. The bioprocessing facility of embodiment 8, wherein the at least one disk stack centrifuge is configured to intermittently discharge accumulated solid particles.
   10. The bioprocessing facility of embodiment 1, wherein the first separator system comprises at least one disk stack centrifuge, wherein the at least one disk stack centrifuge is configured to intermittently discharge accumulated solid particles, and wherein at least a portion of the intermittently discharged accumulated solid particles are in fluid communication with the first defatted emulsion.
   11. A method of separating oil product from one or more stillage compositions in a bioprocessing facility, wherein the method comprises:
separating a first emulsion from a stillage composition, wherein the stillage composition is chosen from thin stillage or a stillage composition derived from thin stillage;
separating separate a first oil product from at least a portion of the first emulsion to form at least a defatted emulsion; and
separating at least defatted, defatted emulsion, and a second oil product or a second emulsion from at least a portion of the defatted emulsion.
   12. The method of embodiment 11, wherein the second emulsion and defatted, defatted emulsion are separated from the at least a portion of the defatted emulsion, and further comprising separating oil product from at least a portion of the second emulsion.
   13. A bioprocessing facility configured to separate oil product from one or more defatted stillage compositions in a bioprocessing facility, wherein the bioprocessing facility comprises:
a source of one or more enzymes and/or a source of one or more microorganisms in fluid communication with the one or more defatted stillage compositions, wherein the bioprocessing facility is configured to combine the one or more enzymes with the one or more defatted stillage compositions and expose the one or more defatted stillage compositions to conditions to enzymatically hydrolyze one or more substrates in the one or more defatted stillage compositions and/or wherein the bioprocessing facility is configured to combine the one or more microorganisms with the one or more defatted stillage compositions and expose the one or more defatted stillage compositions to conditions to convert residual carbohydrate in the one or more defatted stillage compositions into a fermented composition comprising one or more bioproducts; and a separator system in fluid communication with at least a portion of the fermented composition, wherein the separator system is configured to separate at least an oil product or an emulsion from the at least a portion of the fermented composition.
   14. The bioprocessing facility of embodiment 13, wherein the one or more defatted stillage compositions are chosen from defatted syrup, defatted emulsion, and combinations thereof.
   15. The bioprocessing facility of embodiment 13, wherein the at least a portion of the fermented composition is in fluid communication with an evaporator system configured to condense the at least a portion of the fermented composition prior to the separator system.
   16. The bioprocessing facility of embodiment 13, wherein the one or more defatted stillage compositions comprise defatted syrup, wherein the emulsion is a first emulsion, wherein the separator system is a first separator system, and further comprising a second separator system in fluid communication with a source of syrup, wherein the second separator system is configured to separate a second emulsion and the defatted syrup from the syrup.
   17. The bioprocessing facility of embodiment 16, further comprising a third separator system in fluid communication with the first emulsion and the second emulsion, wherein the third separator system is configured to separate a different oil product from at least a portion of the first emulsion and at least a portion of the second emulsion.
   18. The bioprocessing facility of embodiment 16, wherein the first separator system is configured to separate the fermented composition into at least the first emulsion and a defatted fermented composition, wherein at least a portion of the defatted fermented composition is in fluid communication with an evaporator system and/or a dryer system.
   19. The bioprocessing facility of any of embodiments 13-18, wherein the conditions to convert residual carbohydrate into a fermented composition comprising one or more bioproducts comprises anaerobic conditions.
   20. The bioprocessing facility of any of embodiments 13-18, wherein the conditions to convert residual carbohydrate into a fermented composition comprising one or more bioproducts comprises aerobic conditions.
   21. The bioprocessing facility of any of embodiments 13-20, wherein the one or more microorganisms are chosen from at least strain of Candida utilis, at least one strain of Saccharomyces cerevisiae, at least strain of Bacillus subtilis, at least strain of Escherichia coli, at least strain of Aspergillus niger, at least strain of Kluyveromyces marxianus, at least strain of Yarrowia lipolytica, at least strain of Candida tropicalis, at least strain of Candida albicans, and combinations thereof.
   22. The bioprocessing facility of any of embodiments 13-21, wherein the one or more substrates comprise residual carbohydrate.
   23. The bioprocessing facility of any of embodiments 13-22, wherein the one or more microorganisms produce one or more proteins, wherein the fermented composition comprises at least one of the one or more proteins, and wherein at least a portion of the fermented composition is used to form an animal feed product, and/or wherein the one or more microorganisms produce one or more amino acids, wherein the fermented composition comprises at least one of the one or more amino acids, and wherein at least a portion of the fermented composition is used to form an animal feed product.
   24. The bioprocessing facility of embodiment 13, wherein the one or more microorganisms are one or more secondary microorganisms, and further comprising
a primary fermentation system configured to expose a fermentable composition to conditions to ferment monosaccharide via the one or more primary microorganisms to convert monosaccharide into one or more bioproducts and form a first fermented composition, wherein the monosaccharide is derived from hydrolysis of a carbohydrate in biomass;
a system configured to separate one or more bioproducts from the first fermented composition produced in the primary fermentation system and form whole stillage comprising residual carbohydrate; and
a secondary fermentation system configured to expose the one or more defatted stillage compositions and the one or more secondary microorganisms to the conditions to convert the residual carbohydrate in the one or more defatted stillage compositions into the fermented composition comprising the one or more bioproducts.
   25. The bioprocessing facility of embodiment 24, wherein the one or more primary microorganisms are different from at least one of the one or more secondary microorganisms.
   26. The bioprocessing facility of embodiment 25, wherein the one or more primary microorganisms comprise at least one strain of Saccharomyces cerevisiae and the one or more secondary microorganisms are chosen from at least strain of Candida utilis, at least strain of Bacillus subtilis, at least strain of Escherichia coli, at least strain of Aspergillus niger, at least strain of Kluyveromyces marxianus, at least strain of Yarrowia lipolytica, at least strain of Candida tropicalis, at least strain of Candida albicans, and combinations thereof.
   27. The bioprocessing facility of embodiment 24, wherein the one or more secondary microorganisms produce one or more proteins during fermentation, wherein the second fermented composition comprises at least one of the one or more proteins, and wherein at least a portion of the second fermented composition is used to form an animal feed product.
   28. The bioprocessing facility of embodiment 24, wherein the one or more secondary microorganisms produce one or more amino acids during fermentation, wherein the second fermented composition comprises at least one of the one or more amino acids, and wherein at least a portion of the second fermented composition is used to form an animal feed product.
   29. The bioprocessing facility of embodiment 24, wherein the one or more secondary microorganisms comprise at least a first secondary microorganism and a second secondary microorganism, wherein the first secondary microorganism produces one or more byproducts that are consumed by the second secondary microorganism.
   30. A method of separating oil product from one or more defatted stillage compositions in a bioprocessing facility, wherein the method comprises:
combining one or more enzymes with one or more defatted stillage compositions and/or combining one or more microorganisms with the one or more defatted stillage compositions;
exposing the one or more defatted stillage compositions to conditions to enzymatically hydrolyze one or more substrates in the one or more defatted stillage compositions and/or to conditions to convert residual carbohydrate in the one or more defatted stillage compositions into a fermented composition comprising one or more bioproducts; and
separating at least oil product or an emulsion from at least a portion of the fermented composition.
   31. The method of embodiment 30, wherein the emulsion is separated from the at least a portion of the fermented composition, and further comprising separating a different oil product from at least a portion of the emulsion.

What is claimed is:

1. A bioprocessing facility configured to separate oil product from one or more stillage compositions in a bioprocessing facility, wherein the bioprocessing facility comprises:
a first separator system in fluid communication with a source of a stillage composition, wherein the stillage composition is chosen from thin stillage or a stillage composition derived from thin stillage, wherein the first separator system is configured to separate a first emulsion from the stillage composition;
a second separator system configured to separate a first oil product from at least a portion of the first emulsion to form at least a defatted emulsion; and
a third separator system configured to separate at least defatted, defatted emulsion, and a second oil product or a second emulsion from at least a portion of the defatted emulsion.

2. The bioprocessing facility of claim 1, wherein the first separator system is in fluid communication with an evaporator system configured to condense thin stillage into syrup, and wherein the first separator system is configured to separate at least a portion of the syrup from the evaporator system into at least the first emulsion and defatted syrup.

3. The bioprocessing facility of claim 2, wherein the first separator system comprises at least one centrifuge configured to separate at least a portion of the syrup from the evaporator system into at least the first emulsion and the defatted syrup.

4. The bioprocessing facility of claim 2, wherein the second separator system comprises at least one centrifuge configured to separate at least a portion of the first emulsion from first separator system into at least the first oil product and the defatted emulsion.

5. The bioprocessing facility of claim 2, wherein the third separator system configured to separate at least a portion of the defatted emulsion into at least the second emulsion and the defatted, defatted emulsion, wherein at least a portion of the second emulsion is in fluid communication with a fourth separator system configured to separate oil product from at least a portion of the second emulsion, wherein the fourth separator system is the same or different from the second separator system.

6. The bioprocessing facility of claim 1, wherein the first separator system comprises at least one disk stack centrifuge, wherein the at least one disk stack centrifuge is configured to intermittently discharge accumulated solid particles, and wherein at least a portion of the intermittently discharged accumulated solid particles are in fluid communication with the first defatted emulsion.

7. A method of separating oil product from one or more stillage compositions in a bioprocessing facility, wherein the method comprises:
separating a first emulsion from a stillage composition, wherein the stillage composition is chosen from thin stillage or a stillage composition derived from thin stillage;
separating separate a first oil product from at least a portion of the first emulsion to form at least a defatted emulsion; and
separating at least defatted, defatted emulsion, and a second oil product or a second emulsion from at least a portion of the defatted emulsion.

8. A bioprocessing facility configured to separate oil product from one or more defatted stillage compositions in a bioprocessing facility, wherein the bioprocessing facility comprises:
a source of one or more enzymes and/or a source of one or more microorganisms in fluid communication with the one or more defatted stillage compositions, wherein the bioprocessing facility is configured to combine the one or more enzymes with the one or more defatted stillage compositions and expose the one or more defatted stillage compositions to conditions to enzymatically hydrolyze one or more substrates in the one or more defatted stillage compositions and/or wherein the bioprocessing facility is configured to combine the one or more microorganisms with the one or more defatted stillage compositions and expose the one or more defatted stillage compositions to conditions to convert residual carbohydrate in the one or more defatted stillage compositions into a fermented composition comprising one or more bioproducts; and
a separator system in fluid communication with at least a portion of the fermented composition, wherein the separator system is configured to separate at least an oil product or an emulsion from the at least a portion of the fermented composition.

9. The bioprocessing facility of claim 8, wherein the one or more defatted stillage compositions are chosen from defatted syrup, defatted emulsion, and combinations thereof.

10. The bioprocessing facility of claim 8, wherein the at least a portion of the fermented composition is in fluid communication with an evaporator system configured to condense the at least a portion of the fermented composition prior to the separator system.

11. The bioprocessing facility of claim 8, wherein the one or more defatted stillage compositions comprise defatted syrup, wherein the emulsion is a first emulsion, wherein the separator system is a first separator system, and further comprising a second separator system in fluid communication with a source of syrup, wherein the second separator system is configured to separate a second emulsion and the defatted syrup from the syrup.

12. The bioprocessing facility of claim 11, further comprising a third separator system in fluid communication with the first emulsion and the second emulsion, wherein the third separator system is configured to separate a different oil product from at least a portion of the first emulsion and at least a portion of the second emulsion.

13. The bioprocessing facility of claim 11, wherein the first separator system is configured to separate the fermented composition into at least the first emulsion and a defatted fermented composition, wherein at least a portion of the defatted fermented composition is in fluid communication with an evaporator system and/or a dryer system.

14. The bioprocessing facility of claim 8, wherein the one or more microorganisms are chosen from at least strain of Candida utilis, at least one strain of Saccharomyces cerevisiae, at least strain of Bacillus subtilis, at least strain of Escherichia coli, at least strain of Aspergillus niger, at least strain of Kluyveromyces marxianus, at least strain of Yarrowia lipolytica, at least strain of Candida tropicalis, at least strain of Candida albicans, and combinations thereof.

15. The bioprocessing facility of claim 8, wherein the one or more microorganisms produce one or more proteins, wherein the fermented composition comprises at least one of the one or more proteins, and wherein at least a portion of the fermented composition is used to form an animal feed product, and/or wherein the one or more microorganisms produce one or more amino acids, wherein the fermented composition comprises at least one of the one or more amino acids, and wherein at least a portion of the fermented composition is used to form an animal feed product.

16. The bioprocessing facility of claim 8, wherein the one or more microorganisms are one or more secondary microorganisms, and further comprising
a primary fermentation system configured to expose a fermentable composition to conditions to ferment monosaccharide via the one or more primary microorganisms to convert monosaccharide into one or more bioproducts and form a first fermented composition, wherein the monosaccharide is derived from hydrolysis of a carbohydrate in biomass;
a system configured to separate one or more bioproducts from the first fermented composition produced in the primary fermentation system and form whole stillage comprising residual carbohydrate; and
a secondary fermentation system configured to expose the one or more defatted stillage compositions and the one or more secondary microorganisms to the conditions to convert the residual carbohydrate in the one or more defatted stillage compositions into the fermented composition comprising the one or more bioproducts.

17. The bioprocessing facility of claim 16, wherein the one or more primary microorganisms are different from at least one of the one or more secondary microorganisms.

18. The bioprocessing facility of claim 17, wherein the one or more primary microorganisms comprise at least one strain of Saccharomyces cerevisiae and the one or more secondary microorganisms are chosen from at least strain of Candida utilis, at least strain of Bacillus subtilis, at least strain of Escherichia coli, at least strain of Aspergillus niger, at least strain of Kluyveromyces marxianus, at least strain of Yarrowia lipolytica, at least strain of Candida tropicalis, at least strain of Candida albicans, and combinations thereof.

19. The bioprocessing facility of claim 16, wherein the one or more secondary microorganisms comprise at least a first secondary microorganism and a second secondary microorganism, wherein the first secondary microorganism produces one or more byproducts that are consumed by the second secondary microorganism.

20. A method of separating oil product from one or more defatted stillage compositions in a bioprocessing facility, wherein the method comprises:
combining one or more enzymes with one or more defatted stillage compositions and/or combining one or more microorganisms with the one or more defatted stillage compositions;
exposing the one or more defatted stillage compositions to conditions to enzymatically hydrolyze one or more substrates in the one or more defatted stillage compositions and/or to conditions to convert residual carbohydrate in the one or more defatted stillage compositions into a fermented composition comprising one or more bioproducts; and
separating at least oil product or an emulsion from at least a portion of the fermented composition.
